(19) United States
(12) Patent Application Publication
(10) Pub. No.: US 2008/0038710 A1
(43) Pub. Date: Feb. 14, 2008

(54) Assemblies incorporating biomolecules and/or cells with micro-/nanostructures, and methods of making the same for biological applications

(75) Inventors: Ly James Lee, Columbus, OH (US); Yong Yang, Columbus, OH (US); Yubing Xie, Columbus, OH (US); Douglas A. Kniss, Hilliard, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: The Ohio State University, Columbus, OH (US)

(21) Appl. No.: 11/809,787

(22) Filed: Jun. 1, 2007

Related U.S. Application Data
(63) Continuation-in-part of application No. 11/155,808, filed on Jun. 17, 2005.
(60) Provisional application No. 60/809,926, filed on Jun. 1, 2006.
(60) Provisional application No. 60/580,544, filed on Jun. 17, 2004.

(51) Int. Cl. C12N 5/06; A01N 1/00

(52) U.S. Cl. 435/1.1; 435/395

(57) ABSTRACT

Assemblies incorporating cells, biologically active molecules, or combinations thereof. Three dimensional tissue scaffolds are used in the assemblies. The three dimensional tissue scaffolds include a plurality of scaffold layers seeded with cells, biologically active molecules, or combinations thereof, the plurality of cell scaffold layers stacked one on top of another and bonded together in the absence of a solvent or an adhesive. Methods of making the assemblies are also disclosed.

This application claims the benefit of provisional application Ser. No. 60/809,926, entitled Assemblies Incorporating Biomolecules And/Or Cells With Micro-/Nanostructures, And Methods Of Making The Same For Biological Applications, filed Jun. 1, 2006, which is incorporated herein by reference.

This application is a continuation-in-part of U.S. patent application Ser. No. 11/155,808, filed Jun. 17, 2005, entitled Gas Assisted Bonding of Polymers and Polymer Composites, which claims the benefit of provisional application Ser. No. 60/580,544, entitled Supercritical Fluid Assisted Processing and Bonding of Polymers and Polymer Composites, filed Jun. 17, 2004, each of which is incorporated herein by reference.

### BACKGROUND OF THE INVENTION

The present invention relates generally to assemblies incorporating biomolecules and/or cells, and more particularly to assemblies incorporating biomolecules and/or cells with micro-/nanostructures, and to biologically benign and biologically permissive methods of making the assemblies.

Failure or loss of tissues or organs is one of the most critical issues in the medical field. In the United States alone, the population of the patients waiting for organ transplantation has increased by 20% over the past five years, reaching 90,000 by February 2006. Tissue engineering has attracted increased interest over the past 10-15 years as a promising solution to the shortage of donor organs. By applying the tissue-engineering approach, some artificial tissues have been demonstrated successfully and even clinically implanted. Most of these artificial tissues have a simple structure, basically in the layered fashion, and do not have good control on the localization of different cell types. The advancement of tissue engineering necessitates the development of three-dimensional (3-D) culture techniques.

Among the triad of biological tissues, the scaffold plays an essential role. It helps to create 3-D scaffolds with predefined structure to mimic and control the spatial organization of cells and to provide the necessary cues to recapitulate the developmental process in tissue- and organ-specific differentiation and morphogenesis. The commonly used scaffolds, from fibrous matrices to foams, cannot produce well-defined pore structure and cannot localize multiple cell types into specific sites for proper function. Solid free-form fabrication and stereolithography techniques can form well-defined 3-D structures, but their applications are restricted because of their limited control over the shape and dimensions of the building elements (inks or filaments) or their rigorous process parameters. Microfabrication technologies have enabled tissue engineers to better understand cell behaviors and guide cell growth in both single and multicellular organisms. However, it is limited to two-dimensional (2-D), patterned surfaces.

The ability to assemble polymer-based biomedical micro-/nanodevices containing environmentally sensitive biomolecules at low temperatures in order to minimize denaturing is an important consideration. For instance, micro-arrays and microfluidic biochips need to be sealed with a lid, and the construction of three-dimensional tissue engineering scaffolds requires bonding of multiple two-dimensional layers with micro-/nanosized patterns. Processing and bonding of plastic materials usually require the use of either a temperature above the glass transition temperature (T_{g}) of the polymer substrates or organic solvents to deform solid materials so they can be molded into specific shapes or bond the material. For many applications, particularly biomedical products, high temperature and organic solvents may lead to denaturing of biomolecules. Residue of organic solvents is also a severe contamination. In micro- and nanoscale processing, applying heat and solvent alone may not be enough to guarantee high dimensional accuracy during fabrication of micro- or nanoscale features. In addition, heat and solvents tend to deform micro-/nanostructures.

During bonding, polymer interfaces undergo interfacial wetting, diffusion, and randomization stages. Diffusion of polymer chain segments across the interfaces and randomization of polymer chain segments determine the development of bond strength. Bonding of polymers at temperatures below their bulk T_{g} has been studied by several researchers. For example, polystyrene (PS) was bonded at T_{g} −41° C. or 62° C. and poly(2,6-dimethyl 1,4-phenylene oxide) (PPO) was bonded at T_{g} −126° C. or 90° C. using 100 μm thick polymer films. It was believed that this low temperature interfacial bonding resulted from the T_{g} depression in the proximity of polymer surfaces. Experimental observations of polymer thin films have shown that glass transition temperatures near the polymer surface differ from those in the bulk. The competition between the polymer-free surface and the polymer-substrate interactions determines this thickness-dependent T_{g} shift. When the polymer-free surface interaction dominates, the polymer shows a T_{g} depression with reducing thickness near the surface, typically less than 100 nm. An increase in T_{g} is observed as the thickness decreases when a strong interaction exists between the polymer chains and the underlying substrates.

However, literature results show that bond strength develops very slowly below T_{g}. As shown in Table 1, the bond strength of PS/PS at 62° C. with a PC of 0.8 MPa is only 0.08 MPa after 4 h. Even after 24 h, the bond strength is still low, 0.14 MPa, which is unsuitable for practical applications.

In addition, polymers used for dermal drug delivery tend not work because the polymers bend and break.

Therefore, there is a need for well-defined 3-D structures and assembled multiple pre-cultured cell-scaffold constructs into a complex tissue that can faithfully mimic native tissues, such as blood vessels, liver, and kidney, and for methods of making such structures.

### SUMMARY OF THE INVENTION

The present invention meets this need by providing assemblies incorporating biomolecules and/or cells with micro-/nanostructures, and methods of making the same for biological applications. One method includes providing at least two scaffold layers; seeding the scaffold layers with cells, biologically active molecules, or combinations thereof; stacking the at least two scaffold layers; and bonding the at least two scaffold layers using a gas in the absence of a solvent or an adhesive into the three dimensional tissue scaffold.

Another aspect of the invention is a three dimensional cell scaffold comprising: a plurality of cell scaffold layers seeded with cells, biologically active molecules, or combinations thereof, the plurality of cell scaffold layers stacked one on top of another and bonded together in the absence of a solvent or an adhesive.

Another aspect of the invention is a method for incorporating cells, biologically active molecules, or combinations thereof in a scaffold layer. The method includes providing a polymeric scaffold layer; contacting the scaffold layer with cells, biologically active molecules, or combinations thereof; and exposing the scaffold layer and the bioactive molecule to a gas at a biologically permissive temperature, and at a biologically permissive pressure for a sufficient time so that the biologically active molecule diffuses in the scaffold layer.

Another aspect of the invention is a scaffold layer incorporating cells, biologically active molecules, or combinations thereof. The scaffold includes a polymeric scaffold layer having cells, biologically active molecules, or combinations thereof diffused into the polymeric scaffold layer.

Another aspect of the invention is a method of making synthetic tissue. The method includes seeding at least two scaffold layers with cells, biologically active molecules, or combinations thereof; stacking the at least two scaffold layers; bonding the at least two scaffold layers using a gas in the absence of a solvent or an adhesive into a three dimensional scaffold construct; and culturing the three dimensional scaffold construct to form the synthetic tissue.

Another aspect of the invention is synthetic tissue, which includes a three dimensional scaffold construct comprising a plurality of scaffold layers seeded with cells, biologically active molecules, or combinations thereof, the plurality of scaffold layers stacked one on top of another and bonded together in the absence of a solvent or an adhesive, the three dimensional construct cultured to form the synthetic tissue.

Another aspect of the invention is a method of constructing tissue with complex architecture. The method includes seeding at least two scaffold layers with cells, biologically active molecules, or combinations thereof; stacking the at least two scaffold layers; bonding the at least two scaffold layers using a gas in the absence of a solvent or an adhesive into a three dimensional scaffold construct; and culturing the three dimensional scaffold construct to form the complex architecture.

Another aspect of the invention is synthetic tissue having a complex architecture including a three dimensional scaffold construct comprising a plurality of scaffold layers seeded with cells, biologically active molecules, or combinations thereof, the plurality of scaffold layers stacked one on top of another and bonded together in the absence of a solvent or an adhesive, the three dimensional construct cultured to form the complex architecture.

Cells, biologically active molecules, or combinations thereof include, but are not limited to, cells, proteins, DNA, or combinations thereof.

### BRIEF DESCRIPTION OF THE DRAWINGS

FIG. 1A is a graph showing the surface glass transition temperature of PLGA.

FIG. 1B is an SEM micrograph of the side view of a patterned PLGA layer.

FIG. 1C is an SEM micrograph of the side view of a patterned PLGA layer.

FIG. 2A is an SEM micrograph of the top view of a patterned PLGA layer.

FIG. 2B is an SEM micrograph of the side view of a patterned PLGA layer.

FIG. 2C is an SEM micrograph of PLGA layers bonded at 35° C. and 0.79 MPa CO₂ pressure.

FIG. 2D is an SEM micrograph of PLGA layers bonded at 35° C. and 2.17 MPa CO₂ pressure.

FIG. 3A is a graph showing the relationship between the T_{g} data of PLGA and CO₂ bonding performance.

FIG. 3B is an SEM micrograph showing the presence of a porous structure of with submicron-sized cells which was formed within the substrate.

FIG. 4 is SEM micrographs showing PS nanochannels (A) before and (B) after CO₂ bonding at 70° C. and 1.38 MPa CO₂ pressure.

FIG. 5 is a schematic showing the process of assembling a 3D tissue scaffold from microfabricated planar skeletons.

FIG. 6 is a schematic showing the process of bilayer embossing.

FIG. 7A is an SEM micrograph of a top view of a PLGA skeletal layer.

FIG. 7B is an SEM micrograph of a cross-section of bonded PLGA skeletal layers.

FIG. 7C is an SEM micrograph of bonded pure PLGA skeletal layers.

FIG. 8 is a photograph of a 3D scaffold.

FIG. 9 is an SEM micrograph of the cross-section of sealed microchannels on a PS substrate using pure PLGA as the interlayer.

FIGS. 10A and 10B are SEM micrographs showing the cell viability of hMSCs by Calcein AM/EthD-1 stain (green: live cells; red: dead).

FIGS. 10C and 10D are SEM micrographs showing the cell attachment of hMSCs by cytoskeleton F-actin expression.

FIGS. 10E and 10F are SEM micrographs showing the cell proliferation of hMSCs by Brdu-labeling.

FIGS. 11A-11H are SEM micrographs showing the effect of CO₂ pressure on mouse ES cells.

FIG. 12A is a schematic showing the formation of a three dimensional tissue scaffold.

FIGS. 12B and C are confocal microscopic images of the interface between the ES cells/NIH 3T3 cells-scaffold complex and a 3-D view of the assembled cell-scaffold construct with high cellularity.

### DETAILED DESCRIPTION OF THE INVENTION

Using low-pressure carbon dioxide (CO₂), a biologically benign approach to assembling polymeric micro/nanostructures in an aqueous environment in the presence of cells was demonstrated. This method exhibits the ability to integrate multiple cell-scaffolds into a 3-D construct with the potential to develop fully functional tissue substitutes. The CO₂ pressure has little effect on the behaviors of cells studied, including human mesenchymal stem cells from bone marrow (hMSCs), mouse embryonic stem (ES) cells, and NIH 3T3 cells. The method was applied to build multicell tissue layers in biodegradable polymeric scaffolds with well-defined structures. The assembled three-dimensional constructs showed high cell viability and cellularity. It opens a new avenue for tissue engineering, cell therapy, drug delivery and cell-based biochips.

The possibility of using of CO₂ for polymer processing has been extensively explored because CO₂ is nontoxic, nonflammable, and inexpensive. Moreover, CO₂ is an unregulated and non-contaminating processing aid that meets the need for biologically benign processing techniques.

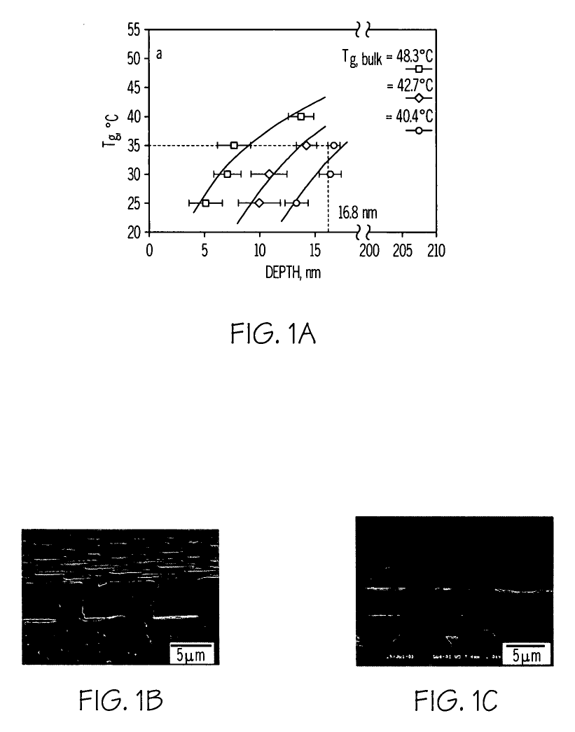

Unlike the fusion of polymers using supercritical CO₂ in previous studies, we found that CO₂ could enhance the chain mobility and reduce the glass transition temperature (Tg) of polymers at the nanoscale under low pressures and below body temperature. This makes it possible to fuse polymeric micro/nanostructures at biologically benign temperatures. As shown in FIG. 1A, Tg near the surface of poly (DL-lactide-co-glycolide) (PLGA) is lower than the bulk Tg under vacuum (leftmost curve), but approaches the bulk value of Tg as distance from the surface increases. The presence of CO₂ does not change this trend, but it greatly reduces the Tg at the same depth and broadens the rubbery layer at the same temperature. These effects increase with CO₂ pressure (middle and rightmost curve). Therefore, the low-temperature polymer nanofabrication techniques can be realized. For instance, a CO₂ pressure as low as 0.79 MPa (114.6 psi) can produce a surface mobile layer with a depth of 16 nm at 35° C. for PLGA (as labeled with the dashed lines in FIG. 1A), making it possible to fuse PLGA structures at low temperatures. Furthermore, CO₂ can diffuse into fluids, such as water, buffer solution, and cultivation media, and can fuse polymer structures in an aqueous environment. In order to explore the abilities of CO₂-assisted fusion of polymeric microstructures in an aqueous environment, PLGA layers were patterned with features as small as 3 μm (see FIGS. 1B (before fusion) and 1C (after fusion)). Several patterned PLGA layers were stacked and held in a container filled with phosphate-buffered saline (PBS) under a compressive pressure of 69 kPa (10 psi) and placed in a pressure vessel at 37° C. After being exposed to CO₂ at 0.69 MPa (100 psi) for 10 min, the multiple PLGA layers were fused into a 3-D construct. The fusion interface was invisible, and the original microstructures were well preserved.

A low-temperature, low-pressure polymer bonding technique based on gas-enhanced chain entanglement near the polymer surfaces was used. The use of a gas can enhance interfacial bonding of micro-/nanostructures at low temperatures without the use of solvents or adhesives. Although not wanting to be limited to theory, it is believed that the presence of CO₂ greatly reduces the surface T_{g}. The bond strength of poly(DL-lactide-co-glycolide) (PLGA) approached 1 MPa with a bonding time of 30 min at 35° C., 0.79 MPa absolute CO₂ pressure.

The layers to be bonded, whether patterned or not, are stacked and placed between two supports. Contact pressure is applied to the assembly to ensure good contact between the layers. The assembly can be placed in a pressure vessel. The gas is introduced into the pressure vessel at a predetermined temperature and pressure, which depend on the materials to be bonded. The assembly remains in the pressure vessel with the gas for a predetermined time, which depends on the materials to be bonded. The pressure is then released, and the assembly is removed from the pressure vessel.

The temperature and gas pressure are selected based on the polymer being bonded. The temperature is selected to be a temperature below the T_{g} of the polymer at the pressure selected. Although this depends on the polymer being used, the temperature is desirably less than 70° C., less than 50° C., or less than 35° C.

The gas pressure is selected based on the polymer and the temperature to be used. The pressure is selected so that the T_{g} of the polymer under the pressure selected is higher than the bonding temperature. Although this varies based on the polymer, it is generally, less than 10 MPa, less than 5, MPa, less than 4 MPa, less than 3 MPa, less than 2 MPa, less than 1 MPa, or less than 0.5 MPa.

The contact pressure is a function of the polymer used, as well as the temperature and pressure. It is generally less than 1 MPa, less than 0.1 MPa, or less than 0.05 MPa. A proper contact pressure helps to achieve interfacial bonding of polymer micro-/nanostructures. The contact pressure is optimized based on the balance of implementing interfacial wetting and maintaining micro-/nanostructures on polymer substrates. An insufficient contact pressure cannot achieve interfacial wetting, while excessive pressure will result in creep deformation during the bonding period and cause micro-/nanostructure deformation.

The gas assisted process can be used to bond polymers to like polymers or to different polymers. For example, poly(lactic-glycolite acid) (PLGA) can be bonded to PLGA using this method. It can also be bonded to polystyrene (PS) and polymethyl methacrylate (PMMA). Suitable polymers include, but are not limited to, PS, PMMA, polycarbonate (PC), PLC, and biodegradable polymers such as PLGA, and polylactic acid (PLA).

The process can also be used to bond polymers to non-polymeric materials. Suitable non-polymeric materials include, but are not limited to metals, silicon, and silicon oxide (usually coated with a polymer thin layer).

CO₂ is a desirable gas for polymer processing since it is non-toxic, nonflammable, and inexpensive. After processing, CO₂ removal from the polymer can be accomplished simply by depressurization of the system. We have found that carbon dioxide can serve as a good processing aid for polymers including, but not limited to, PS, PMMA, PC, and biodegradable polymers such as PLGA. For example, CO₂ can be used as a processing aid for PLGA at room temperature (or lower temperature) and lower pressures (<0.5 MPa). Under such conditions, PLGA containing biomolecules can be processed or bonded without applying heat or organic solvents. This technique can be applied to other polymers, such as PS and PMMA, although the processing temperature and pressure may vary from case to case.

Although carbon dioxide is the most desirable choice for gas-assisted processing and bonding, other gases including, but not limited to, methane, ethane, water, nitrogen, oxygen, chlorofluorocarbon (CFC), or combinations thereof may also be used if desired. For example, methane and ethane work much better than carbon dioxide for polyolefins such as polyethylene and polypropylene.

Nano-sized or micron-sized particles may be used to provide dimensional stability for the polymer. For example, when a small amount of nanoparticles such as organoclay is added to the PLGA, dimension integrity can be maintained very well in the bonding process.

Carbon dioxide has been used to bond micro- and nano-patterned polymer layers for the fabrication of 3D tissue scaffolds. The same technique can be used for the bonding and assembly of biochips/biosensors, drug delivery devices, and any polymer based microelectromechanical systems (MEMS)/nanoelectromechanical systems (NEMS) devices.

### EXAMPLE 1

CO₂-Enhanced 3D Assembly of Polymer Microstructure

An array of wells 5 μm in diameter and 3.9 μm in height was fabricated on the PLGA nanocomposite via photolithography and microembossing. The PLGA nanocomposite was prepared by mixing PLGA with organoclay (Cloisite 30B from Southern Clay Products, Inc.) (95/5 wt %) at 120° C. and 150 rpm for 5 minutes using a microcompounder (DACA Instruments). During CO₂-enhanced assembly at the microscale, only the PLGA nanocomposite was used. For simplicity, PLGA refers to the PLGA nanocomposite unless otherwise indicated.

The pattern was first generated on a photosensitive PMMA coated on a Si wafer by conventional photolithography. Then, the mixture of poly(dimethylsiloxane) (PDMS) resin and curing agent (SYLGARD 184 from Dow Coming) was poured onto the patterned mold. After 4 hours of curing at 60° C., the PDMS mold with inverse pattern was peeled off. The PLGA thin film (˜100 μm) was prepared by compression molding. The film was set at 140° C. for 1 minute, and a pressure of 0.07 MPa was used to emboss the PDMS mold into the molten PLGA film. After 1.5 minutes, ice water was used to cool down the system. When the temperature was below 38° C., the patterned PLGA layer was peeled off from the PDMS mold. The total operation time was 2.5 minutes.

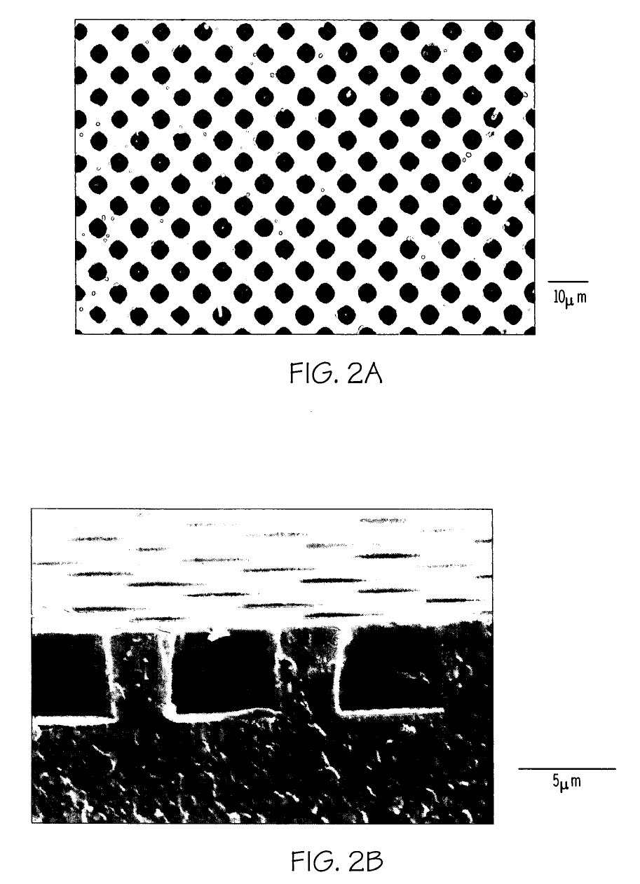

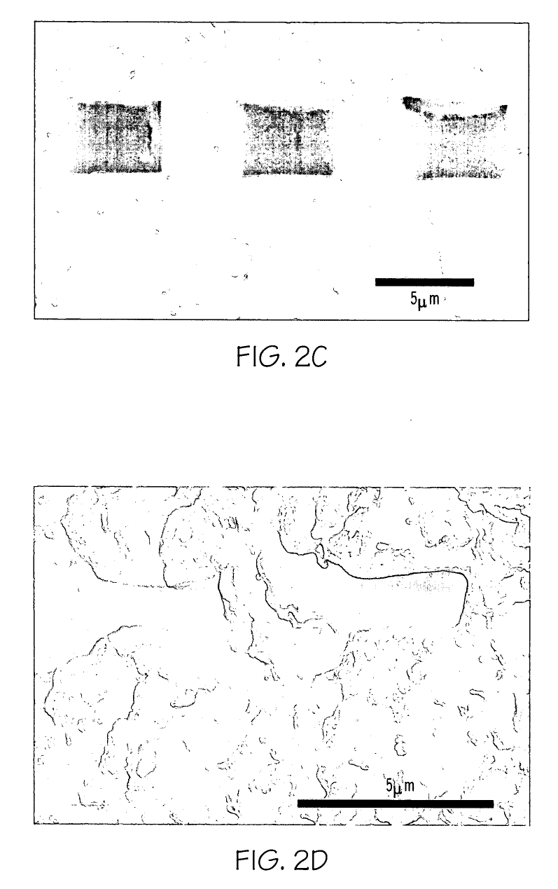

FIG. 2 represents the typical case of CO₂ bonding of PLGA microstructures. FIGS. 2A and 2B show the top view and side view of the patterned PLGA layer. Each of the micro-wells has a diameter of 5 μm and a height of 3.9 μm. Multiple layers of patterned PLGA were stacked to form a laminated assembly. A contact pressure of 0.06 MPa was exerted on the assembly, which ensured good contact between the PLGA layers. The assembly was placed in the pressure vessel, and saturated with CO₂ at 35° C. and 0.79 MPa for 4 hours, after which the pressure was quickly released. The result was a multi-layer assembly bonded into a single piece. The cross-section profile of the bonded layers was inspected using SEM, and the bonding interface was invisible, as shown in FIG. 2C. The original height of the wells was 3.9 μm, while the height after bonding was 3.95 μm. The well diameter remained 5 μm after bonding. Thus, the microstructures were retained. When the CO₂ pressure increased to 2.17 MPa with other parameters maintained constant, the micro-wells shrank because of foaming, as shown in FIG. 2D.

The cross-section profiles of bonded layers were measured using a Hitachi S-3000H scanning electron microscope (SEM). The PLGA samples were frozen in liquid nitrogen for 20 minutes and cut through the bonded layers using a microtome knife, which was frozen at the same condition as the samples. The PLGA samples (without fracture) were sputter-coated with gold at an argon pressure of 14 Pa for 30 seconds at a current of 7 mA. This step was repeated 6 times over 60-second intervals to prevent the samples from overheating.

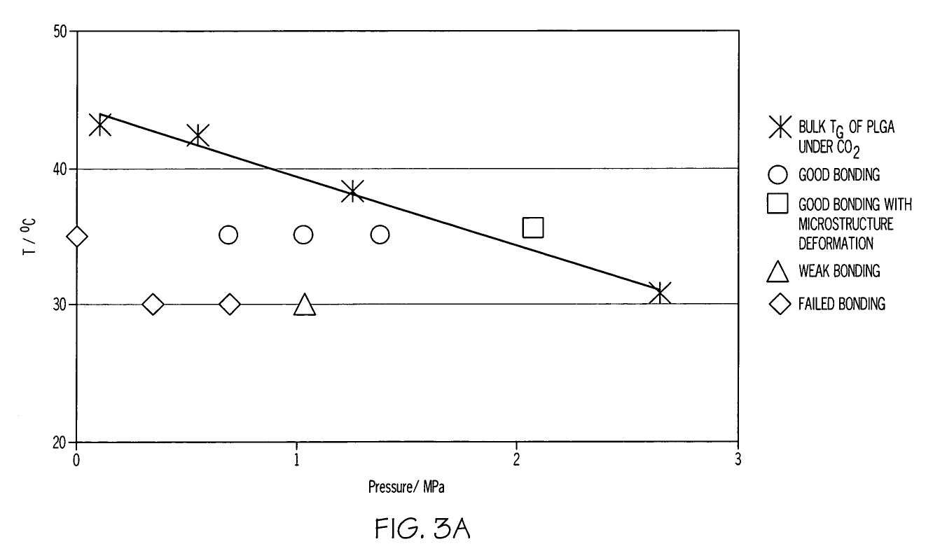

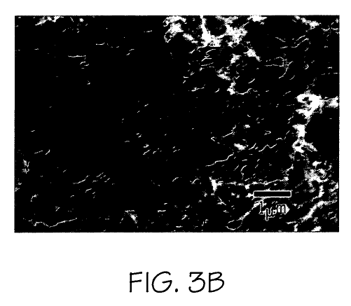

FIG. 3A shows the relationship between CO₂ bonding performance and the T_{g} of PLGA. The T_{g} of PLGA under CO₂ was determined using a differential scanning calorimeter (DSC2920, TA) equipped with a high-pressure cell, using the heating rate of 10 K min⁻¹. The asterisk indicates the bulk T_{g} of PLGA under CO₂ pressures. The solid line represents the regression line of these bulk T_{g}. Circles indicate good bonding; squares, good bonding with microstructure deformation; triangles, weak bonding; and diamonds, failed bonding.

The best bonding performance occurred when the T_{B} was slightly lower than the bulk T_{g}: good bonding was achieved, and the surface microstructures maintained their integrity. For example, good bonding was achieved at 35° C. and 0.79 MPa for 4 hours. When the T_{B} was far below the bulk T_{g}, there was no bond strength. There was no bonding in the absence of CO₂ when other parameters remained the same, even after 24 hours. When the T_{B} was higher than the bulk T_{g}, visible deformation of microstructures was observed. For example, when the CO₂ pressure was 2.17 MPa, the bulk T_{g} of PLGA (34° C.) was below the bonding temperature of 35° C. SEM observation revealed that a porous structure with submicron-sized cells was formed within the substrate (FIG. 3B), resulting in deformation of the micro-wells. CO₂ enhanced chain motion at the polymer surface causes the completion of the interface bonding. At points far below the bulk T_{g}, the motion of macromolecular segments enhanced by CO₂ at the surface is not sufficient to realize effective bonding. When the T_{B} is above the bulk T_{g}, the segments of macromolecular chains, both near the surface and in the bulk, can move, causing microstructure deformation.

To avoid foaming-caused microstructure deformation, the bonding temperature should be slightly lower than the bulk T_{g}.

### EXAMPLE 2

CO₂-Enhanced 3D Assembly of Polymer Nanostructure

Nano-sized channels with a spectrum of widths from 100 nm to 600 nm were patterned on a negative tone resist AZPN114 coated on a Si wafer using electron beam lithography (EBL). The PDMS mold with inverse nano-sized pattern was obtained by following the procedure described above, except that the PDMS mixture was cured for 24 hours at room temperature. A PS film (˜50 μm, prepared by compression molding) was set at 220° C. for 1 minute, and a pressure of 0.1 MPa was used to emboss the PDMS mold into the molten PS film. After 30 s, ice water was used to cool down the system. When the temperature was below 40° C., the PS nanochannels were obtained by peeling off from the PDMS mold. The total operation time was 1.5 minutes. Another PS film (˜2 μm) was generated as the lid by spin-coating 5 wt. % PS toluene solution on a pre-cleaned glass slide. The films with the slides were floated onto a pool of deionized water and captured on a cured PDMS plate. The films with the PDMS plate were then dried in a vacuum for 24 h at 90° C. to remove residual water.

The PS lid on the PDMS plate was aligned onto the patterned PS layer. The aligned sample was placed between two glass slides, and a contact pressure was applied on the sample. The whole assembly was placed in the pressure vessel for CO₂ bonding as described above.

At the nanoscale, the surface roughness is comparable to the nano-sized features. In order to obtain the desired intimate contact between the lid and the nanochannels, the PS lid was transferred onto a PDMS plate and pressed into intimate contact with the top surface of the nanochannels. Intimate contact could be obtained because of the elastomeric nature of PDMS. A contact pressure of 0.28 MPa achieved good interfacial wetting without deforming the nanostructure.

The cross-section profiles of bonded layers were measured using a Hitachi S-3000H SEM. The PS samples were frozen in liquid nitrogen for 20 minutes and the lid was broken using tweezers in a liquid nitrogen environment. The PS samples (without fracture) were sputter-coated with gold at an argon pressure of 14 Pa for 30 seconds at a current of 7 mA. This step was repeated 6 times over 60-second intervals to prevent the samples from overheating.

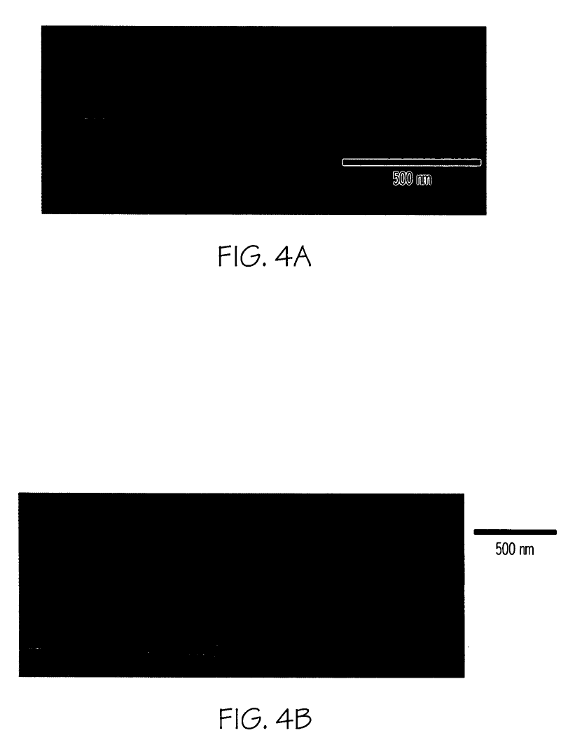

FIG. 4 shows the SEM micrographs of PS nanochannels before (A) and after (B) bonding at 70° C., 1.48 MPa CO₂ pressure. Compared with PS nanochannels before bonding, the nanostructures, including surface roughness, were well preserved after CO₂ bonding. The bonding temperature of 70° C. is 26° C. below the bulk T_{g} of PS (96° C.) under 1.48 MPa CO₂ pressure.

### EXAMPLE 3

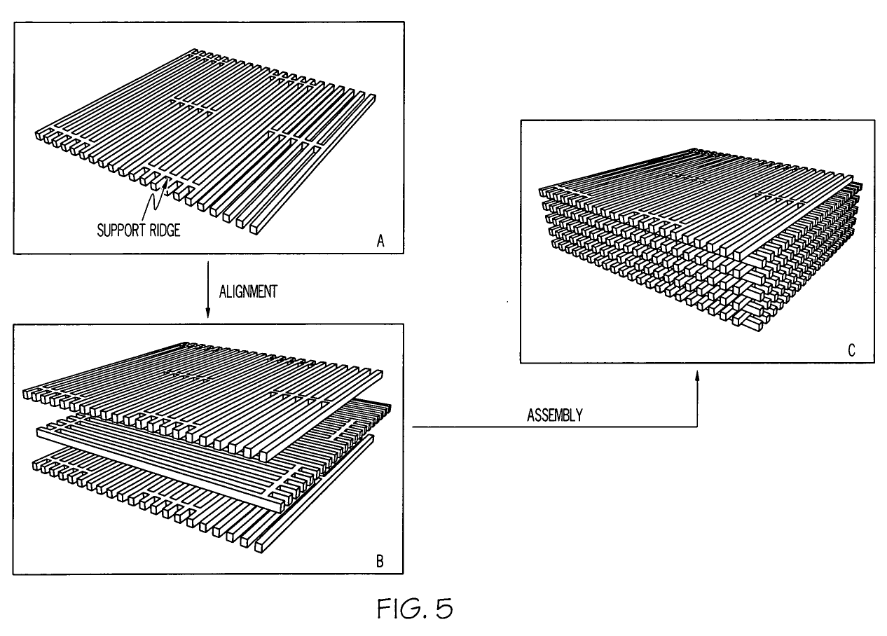

Generally, the process to fabricate the well-defined 3D scaffold involves three stages: 1) photolithography to generate the mold with the planar skeletal structure; 2) microembossing to fabricate 2D scaffold skeletons with the planar structure transferred from the mold; and 3) CO₂ bonding to assemble the laminated multiple 2D skeletons into 3D tissue scaffolds. FIG. 5 shows the design concept and the process to assemble single-layer scaffold skeletons into multiple layers of the 3D architecture with a prescribed pore structure.

The skeletal structure was generated via photolithography and microembossing. Photolithography involved photomask fabrication, wafer cleaning, spin coating of the photoresist, soft baking, UV-exposure, post-exposure baking, and developing. To make the photomask, FreeHand (a computer-aided design software) was used to create the micropattern, which was then printed as transparent lines (60 μm in width with 120 μm in spacing between two parallel lines) on a black background on a transparency using a high-resolution laser printer (3386 dpi). A silicon wafer, which was pretreated with isopropyl alcohol, rinsed with deionized water and then dehydrated, was spin-coated with a thin film (60 μm in thickness) of an epoxy photoresist (SU-8 100 from MicroChem Corp.) using a spin coater (Model P6700 from Specially Coating Systems Inc.). This was followed by soft baking for 8 minutes at 65° C., and then hard baking for 25 minutes at 95° C. The SU-8 layer was exposed to the UV light (350-400 nm) on a Cobalt mask aligner. After exposure and post-exposure-baking, the SU-8 layer on the wafer was immersed in the SU-8 developer to produce the SU-8 mold with all the skeletal features.

The mixture of poly(dimethylsiloxane) (PDMS) resin and curing agent (SYLGARD 184 kit, Dow Corning) in a 10:1.05 w/w ratio was poured onto the SU-8 mold. After curing for 2 h at 65° C., an inverse PDMS mold was peeled off from the SU-8 mold.

The bilayer microembossing process (FIG. 6) was developed to produce scaffolds with open channels. The PLGA thin film (˜100 μm) was prepared via compression molding. A PDMS mold with a PLGA film on the top was set at a temperature higher than 140° C. for 1 min, and a pressure of 0.5 MPa was applied to press another PDMS mold face-down into the molten PLGA film in an orthogonal orientation to the former PDMS mold. After 1.5 min holding, ice water was used to cool down the system. When the temperature was below 38° C., the PLGA bilayer was peeled off from the PDMS molds. The total operation time was 2.5 min.

Multiple PLGA scaffold skeletons were stacked in an orthogonal orientation and placed between two glass slides. A contact pressure of 0.06 MPa was applied by exerting predetermined weight on the assembly. At 35° C., an ISCO 500C high-pressure syringe pump was used to deliver and control the CO₂ pressure at 0.79 MPa in a pressure vessel. After saturation with CO₂ for 1 h, the pressure was quickly released, and the bonded assembly was taken out from the vessel. To evaluate the bonding performance, the bonded skeletons were frozen in liquid nitrogen for 20 minutes and cut through the bonded layers using a microtome blade, which was kept at the same freezing temperature as that of the samples. The cross section cut-through samples were observed under a scanning electron microscope.

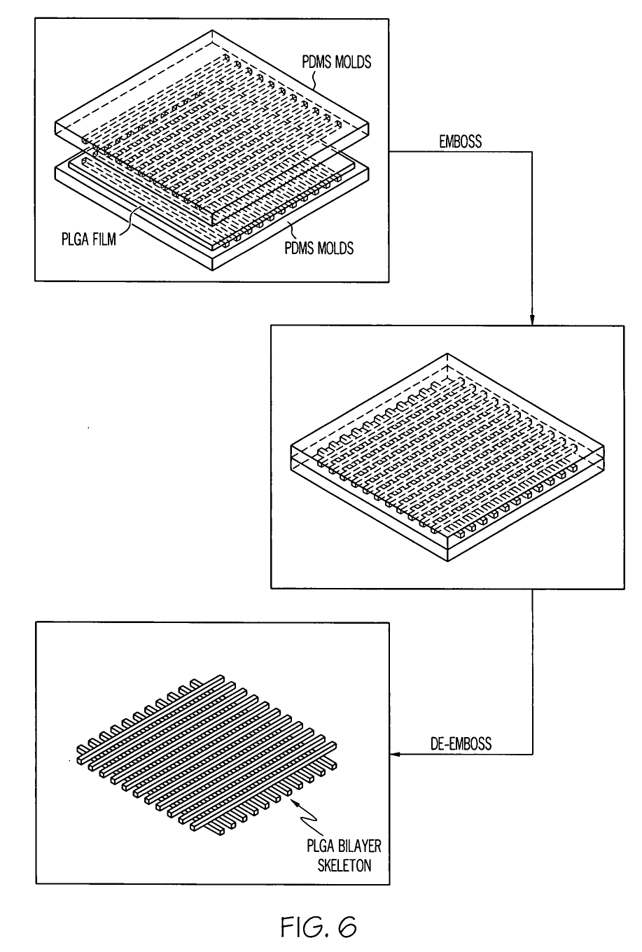

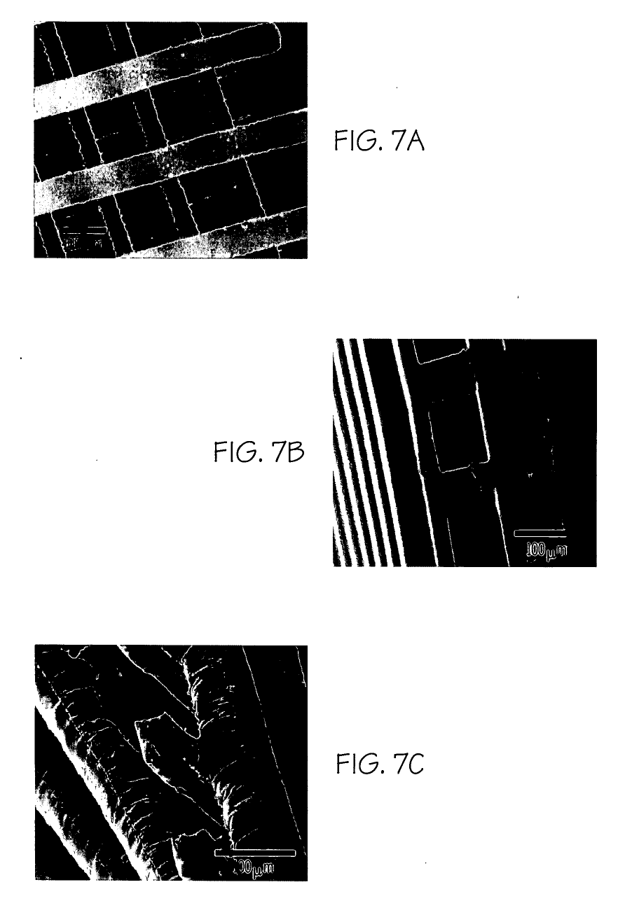

FIG. 7A shows the top view of a PLGA bilayer skeletal scaffold. The cross-section profile of the bonded layers was inspected using SEM as shown in FIG. 7B. The lamination was bonded seamlessly, and excellent microstructure preservation was obtained. When pure PLGA was used, obvious deformation of microstructures was observed. The original rectangular shape of the ridge was lost after bonding (FIG. 7C). The presence of the nanoscale clay particles improved the dimensional stability, which may be due to the formation of a percolated network structure resulting from randomly oriented nanoscale clay particles. FIG. 8 shows a large scaffold consisting of 320 layers of skeletons, each layer having the dimensions of 10 mm×10 mm×60 μm.

A novel CO₂ bonding technique was developed for 3D assembly of polymer micro-/nanostructures at low temperatures. Precise replication at a scale as small as 10 nm was achieved by embossing PDMS molds into molten polymer substrate. Bilayer embossing technique is likely to be commercially viable for continuously manufacturing scaffold skeletons by applying roller hot embossing. The CO₂ bonding technique was demonstrated to be a powerful method to assemble 3D polymer scaffolds at low temperatures. More importantly, there is no organic solvent involved throughout the whole process from pattern design to the final 3D scaffold. This new micro-/nanofabrication process provides a low-cost, fast, solvent-free method to construct 3D scaffolds with predefined structure, and should be viable for commercial application.

The lap-shear measurements were conducted according to ASTM 3163(01) at a crosshead speed of 0.5 mm/min with the modified joint geometry. The specimen dimension was 0.55 mm thickness, 12.7 mm width, and 50 mm length. The overlapped area was 6.4×12.7 mm². The distance between the jaws was 68.2 mm, with the joint located in the middle.

The bond strength between PLGA layers was over 1 MPa after 4 h at 35° C. (below body temperature) under 0.79 MPa CO₂ pressure, as determined by the lap-shear measurement (during the lap-shear measurements, the PLGA specimens yielded before any de-bonding in the overlap area, indicating a bond strength higher than 1 MPa, the yield stress of PLGA). Without the presence of CO₂ pressure, there was no bond strength observed at 35° C., even after 24 h. In addition, the lap-shear measurements were conducted on the CO₂-bonded samples after different bonding times, i.e., 10, 30, 60, 90, 120, and 240 min, when other parameters were maintained as constants. It was found that there was bond strength developing after a bonding time of 10 min. The bond strength approached 1 MPa after 0.5 h. This bond strength was strong enough to maintain the 3D structure. Even after being used to culture cells in liquid media for four days, the scaffold still maintained its structural integrity.

TABLE 1
Comparison of bond strength from lap-shear measurements

| | | | | | Bond |
|---|---|---|---|---|---|
| | T_{B} | P_{B} | P_{C} | t_{B} | Strength |
| | [° C.] | [MPa] | [MPa] | [hrs] | [MPa] |
| PLGA/PLGA | 35 | 0.69 | 0.06 | 4 | >1.00 ± 0.05 |
| PMMA/pure PLGA/ | 35 | 0.69 | 0.06 | 4 | 0.32 ± 0.08 |
| PMMA | | | | | |
| PS/pure PLGA/PS | 35 | 0.69 | 0.06 | 4 | 0.30 ± 0.04 |
| PS/PS [a] | 62 | N/A [b] | 0.8 | 4 | 0.08 ± 0.03 |
| PPO/PPO [a] | 90 | N/A [b] | 0.8 | 4 | 0.03 ± 0.01 |

[a] The bond strength for PS/PS and PPO/PPO are literature values.
[b] No CO₂ was applied.

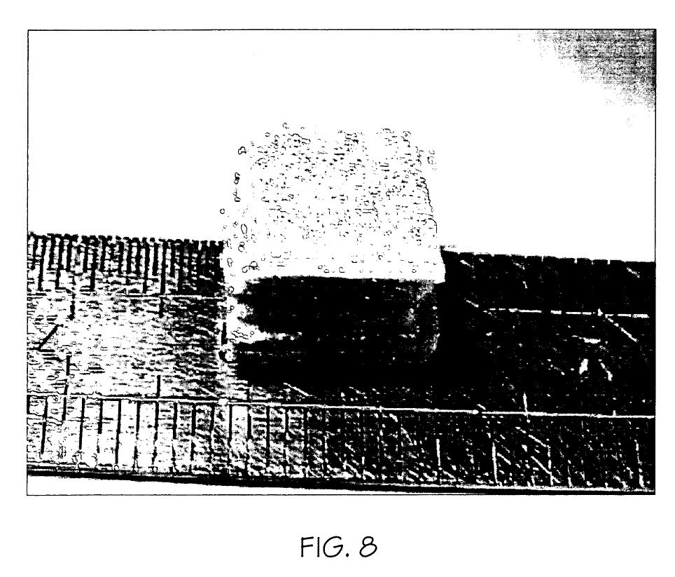

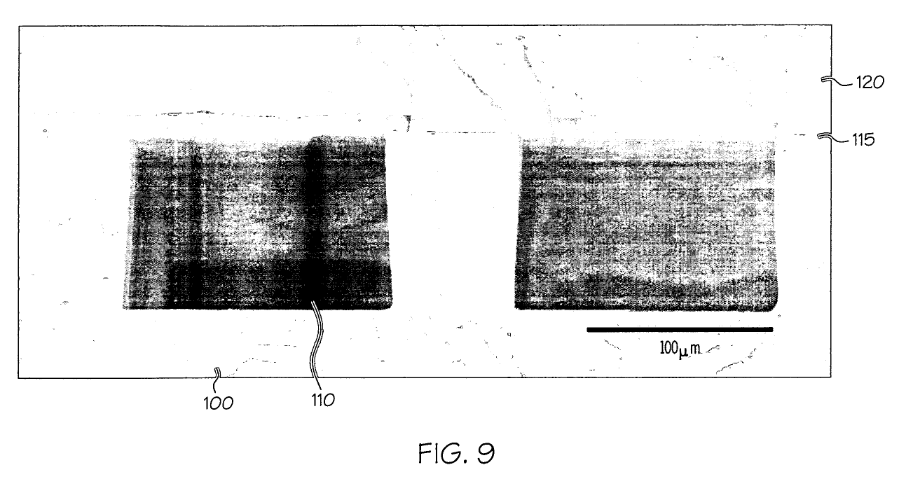

Bonding other thermoplastics, such as PS, PMMA, and polycarbonate, can be achieved at higher bonding temperature and CO₂ pressure. For instance, a good bonding of PS/PS was achieved at 50° C. and 3.4 MPa CO₂ pressure. If pure PLGA is used as an interlayer, PMMA/PMMA and PS/PS bonding can be achieved at the same conditions as for bonding of materials similar to PLGA (i.e., 35° C. and 0.69 MPa CO₂ pressure). The bond strength is up to 0.32 MPa for PMMA and 0.30 MPa for PS as shown in Table 1. This provides a way to seal microfluidic biochips below body temperature. FIG. 9 shows a cross-section of sealed microchannels 10 on a PS substrate 100 using pure PLGA thin film 115 as an interlayer to join the PS lid 120 and PS microchannels 110.

### EXAMPLE 4

The issue of whether the cells can survive under the CO₂ pressure applied during construction of the 3D tissue scaffold was investigated. Human mesenchymal stem cells (hMSCs) and mouse embryonic stem (ES) cells were used as model cell lines. hMSCs were provided by Tulane University. hMSCs were grown in a maintenance medium, consisting of Minimum Essential Medium Alpha Medium (αMEM) supplemented with 16.5% (v/v) FBS, 100 U/ml penicillin, 100 μg/ml streptomycin, and 2 mM L-glutamine (Invitrogen, Carlsbad, Calif.). The CCE ES cell line was provided by StemCell Technologies, Inc. (Vancouver, Canada). The cells were grown on gelatin-coated tissue culture flasks in a maintenance medium consisting of Dulbecco's Modified Eagle's Medium (DMEM with 4.5 g/L D-glucose) supplemented with 15% (v/v) fetal bovine serum (FBS), 100 U/ml penicillin, 100 μg/ml streptomycin, 0.1 mM non-essential amino acids, 10 ng/ml murine recombinant leukemia inhibitory factor (IIF; StemCell Technologies), 0.1 mM monothioglycerol (Sigma Aldrich), 2 mM L-glutamine, and 1 mM sodium pyruvate (Invitrogen). NIH 3T3 cells were grown in DMEM/F12 supplemented with 10% (v/v) newborn calf serum (NCS; Invitrogen), 2 mM L-glutamine, and 1 mM sodium pyruvate.

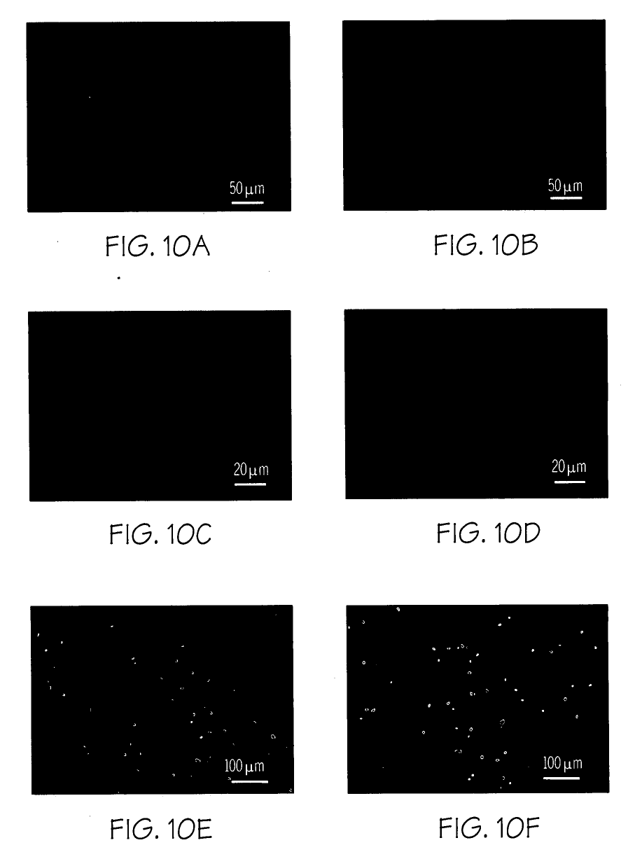

hMSCs were placed in a pressure vessel, and CO₂ was injected at a rate of 0.28 MPa (40 psi) per min and maintained at 1.38 MPa (200 psi) for 30 min, followed by very slow releasing of CO₂ pressure at 0.05 MPa (6.7 psi) per min. hMSCs were viable and remained attached to the substrate surface after the CO₂ exposure (comparing FIG. 10A with 10B—green: live cells; red: dead cells). This indicates that greater than 99% hMSCs were viable following CO₂ treatment, even when the pH dropped from 7.4 to 6.5 during pressurization (the process varied from 15 min to 2 hrs). The slow and constant depressurization reduced, and even eliminated, the bubble formation, which may cause cell detachment and loss. The cell integrity was evaluated by examining the organization of the actin cytoskeleton. In both CO₂ treated and untreated cells, a pattern of F-actin distribution within the cytoplasm that was consistent with cells spread onto a surface under static culture conditions (comparing FIG. 10C with 10D). The incorporation of bromodeoxyuridine (BrdU) into hMSCs was measured as an index of proliferative potential (comparing FIGS. 10E with 10F). The percentage of proliferating hMSCs after CO₂ treatment is around 70%, which is the same as the cells without CO₂ treatment (data not shown). This illustrates that CO₂ has no obvious effects on the viability, attachment, or the proliferation of hMSCs, which is important to the differentiation and function of hMSCs.

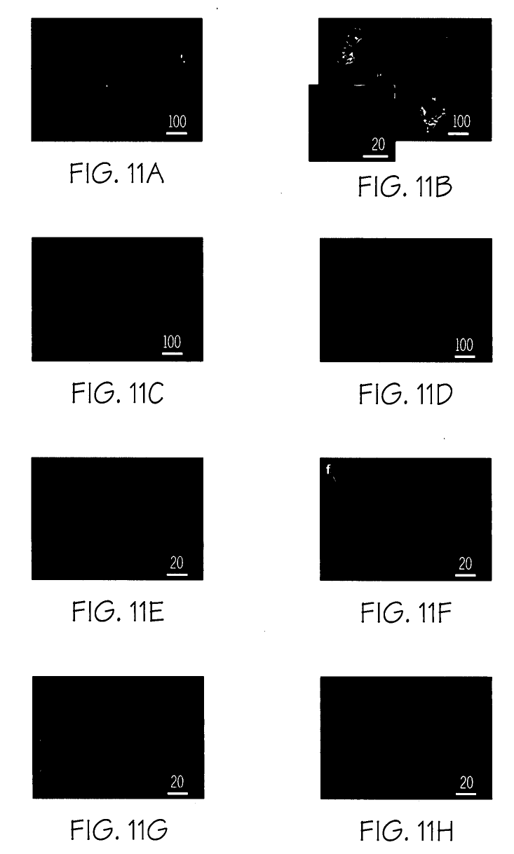

ES cells can survive the CO₂ exposure at 1.38 MPa (200 psi) as well. After the CO₂ exposure, the ES cells were alive, and the size of the cell colony increased after it was cultured in the maintenance medium for 1 day (FIG. 11A—green: live cells; red: dead cells). All ES cells expressed Oct-4 in nuclei (FIG. 11B), the canonical marker of pluripotency in ES cells. This demonstrates that all cells that survived the CO₂ exposure were well maintained in an undifferentiated state. After being cultured in the medium without a leukemia inhibitory factor (LIF), the ES cells that experienced CO₂ exposure formed embryonic bodies (EB bodies) (FIG. 11C—green: live cells; red: dead cells), a necessary stage for differentiation, at a rate similar to the untreated cells. Under further culture, the outgrowth of the EB bodies differentiated spontaneously into multiple lineages, revealed by the expression of marker genes of mesoderm (BMP-4; FIG. 11D), neuroectoderm (Pax6; FIG. 11E), neuronal cells (nestin; FIG. 11F), cardiac muscle cells (Nkx2.5; FIG. 11G), and skeletal muscle cells (Myf5; FIG. 11H). This demonstrated that the ES cells maintained their differentiation potential for early embryonic lineages, neurogenesis, cardiogenesis, and myogenesis after CO₂ exposure. Thus, the CO₂ pressure has little effect on the pluripotency and differentiation potential of mouse ES cells.

### EXAMPLE 5

The finding that the CO₂ pressure had little effect on the behaviors of hMSCs and ES cells led to further investigation of the feasibility of integrating the different types of cells grown in multiple PLGA scaffolds into a 3-D complex cell-scaffold construct using CO₂-assisted bio-assembly.

Microstructures were patterned and fabricated into PLGA films by using photolithography and micro-embossing. Multiple patterned PLGA layers were stacked and placed in the media. A contact pressure was applied by exerting a predetermined weight on the sample. The whole assembly was placed in a pressure chamber. At 37° C., an ISCO 500C high-pressure syringe pump was used to deliver and control the CO₂ pressure in the chamber. The CO₂ pressure was increased to 0.55 MPa (80 psi) at an approximate rate of 0.1 MPa (15 psi) per min, and maintained at 0.55 MPa for 10 min, followed by a slow-pressure release at 0.02 MPa (3 psi) per min. Furthermore, the well-defined PLGA scaffolds were put in a 24-well plate and sterilized using UV light in a tissue culture hood.

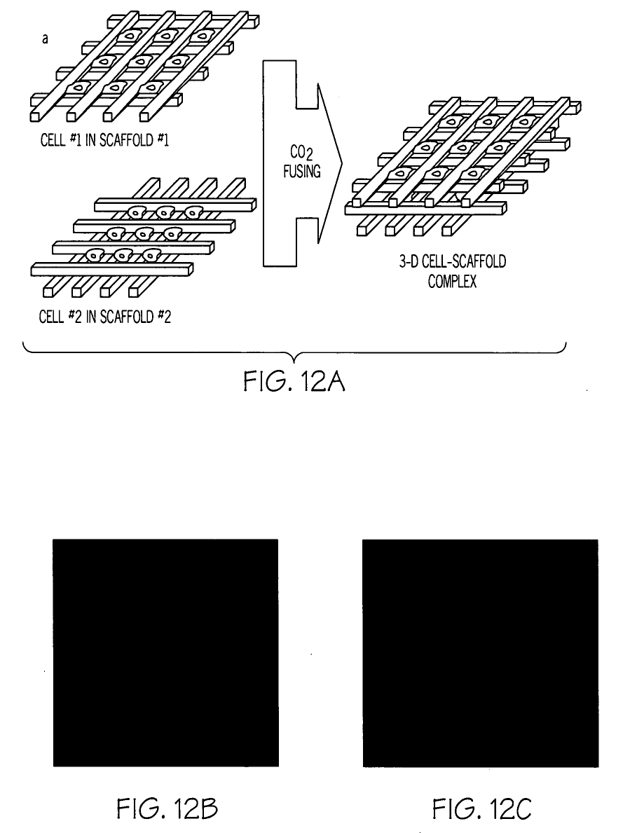

Mouse ES cells were seeded on the PLGA scaffolds and grown for 4 days. NIH 3T3 cells were seeded on the PLGA scaffolds and grown for 1 day. These PLGA scaffolds containing cells were stacked layer-by-layer (as illustrated in FIG. 12A) and placed in a sterile container with the culture medium, and a contact pressure of 55 kPa (8 psi) was applied. The whole system was placed in the pressure chamber for CO₂ fusion using the aforementioned process.

This construct was stained by calcein AM to show the cell viability and cell distribution. Cells were cultured on coverslips or PLGA scaffolds fitted in a 24-well plate overnight. The coverslips or PLGA scaffolds were transferred to 35 mm tissue culture dishes or sterile containers with the culture medium and placed inside the CO₂ chamber. After exposure to CO₂ under 1.38 MPa (200 psi) at 37° C. for 45 min following the aforementioned pressure increase and release rates, the coverslips or PLGA scaffolds were transferred to a 24-well plate and cultured with fresh culture medium. The cells were stained with Calcein AM/Ethidium homodimer-1 (EthD-1; Molecular Probes, Eugene, Oreg.) working solution, composed of 4 μM EthD-1 and 4 mM Calcein AM in PBS, for 30 min at room temperature. The labeled cells were viewed under the epifluorescence microscope (Nikon TE200, Tokyo, Japan) using the FITC and Rhodamin filter sets, where the green fluorescence indicated the live cells labeled by Calcein and the red fluorescence indicated the dead cells labeled by EthD-1.

The cell morphology and scaffold structure of the assembled 3-D construct was revealed under confocal laser scanning microscopy (FIGS. 12B and 12C—green: live cells; red: dead cells). The green fluorescence revealed that the cells in the 3-D construct were alive after CO₂-assisted bio-assembly. The ES cells (round shapes) and the NIH 3T3 fibroblasts (stretched shapes) were controlled in the pre-designated sites in the well-defined 3-D structure.

This approach is not restricted to hierarchical tissues. It can be used to construct heterogeneous but well-organized 3-D tissues as well. Multiple cells can be seeded and cultured into cell-specific polymeric scaffolds with pre-defined structures, and these cell-scaffold building blocks can be coordinated into a large-scale system using a precise alignment and manipulation tool. They can then be assembled into a complex construct using the CO₂-assisted assembly technique. Moreover, signaling molecules can be integrated into the polymeric scaffold blocks selectively before cell seeding. Therefore, the controlled and sustained delivery of the signaling molecules at the specific location can be realized in the complex, multi-functionalized tissue.

This CO₂-assisted bio-assembly method is a highly affordable and biologically benign process, particularly for the simultaneous assembly of a large number of micro/nanostructures containing temperature- and/or solvent-sensitive biomolecules and cells. In addition to tissue engineering, it can have wide applications in polymer-based bioMEMS/NEMS, such as in packaging of micro/nanoscale biochips and drug delivery devices with pre-loaded cells and biomolecules.

### EXAMPLE 6

Combination of Microembossing with CO₂ Bonding for Cell and/or Biomolecular Laden Structure Formation.

Microstructures would be patterned and fabricated into PLGA films by using photolithography and micro-embossing (Yong Yang, et al., Biomaterials, 2004, which is incorporated herein by reference). Biomolecules would be incorporated in scaffolds by CO₂ impregnation. Cells would be seeded on the well-defined PLGA scaffolds. They would be grown to form desired tissue-like structures. Scaffolds containing cells and scaffolds containing biomolecules would be stacked layer-by-layer and placed in a sterile container with the culture medium. An appropriate contact pressure would be applied (e.g., about 55 kPa (8 psi)). The whole system would be placed in the pressure chamber for CO₂ fusion. At about 37° C., an ISCO 500C high-pressure syringe pump would be used to deliver and control the CO₂ pressure in the chamber. The CO₂ pressure would be increased to an appropriate amount (e.g., about 0.69 MPa (100 psi)) at an appropriate rate (e.g., approximately 0.14 MPa (20 psi) per min), and maintained at an appropriate level (e.g., about 0.69 MPa) for an appropriate length of time (e.g., about 10 min), followed by a slow-pressure release (e.g., about 0.02 MPa (3 psi) per min). After CO₂ fusion, the assembled cell-scaffold constructs would be cultured to form 3D constructs.

This process provides well-defined biomimic structure for tissue morphogenesis, biosensing, or drug delivery.

### EXAMPLE 7

Methods of Synthetic Tissue Formation Using Multiple Cell Lineages and Natural Differentiating

Stem cells (embryonic stem (ES) cells or mesenchymal stem cells) were seeded on the PLGA scaffolds. They were grown in differentiation medium for 20 days to form multiple cell lineages, respectively. These scaffolds containing cells were stacked layer-by-layer and placed in a sterile container with the culture medium. An appropriate contact pressure was applied (e.g., about 55 kPa (8 psi)). The whole system was placed in the pressure chamber for CO₂ fusion. At about 37° C., an ISCO 500C high-pressure syringe pump was used to deliver and control the CO₂ pressure in the chamber. The CO₂ pressure was increased to an appropriate level (e.g., about 0.69 MPa (100 psi)) at an appropriate rate (e.g., approximately 0.14 MPa (20 psi) per min), and maintained at an appropriate level (e.g., about 0.69 MPa) for an appropriate length of time (e.g., about 10 min), followed by a slow-pressure release (e.g., about 0.02 MPa (3 psi) per min). After CO₂ fusion, the assembled cell-scaffold constructs were cultured and undergo natural differentiating to form tissue-like structures.

The tissue construct has controlled 3-D configurations of multiple cell types with in vivo-like function.

### EXAMPLE 8

Methods of Constructing Tissues and Organs with Complex Architecture

The PLGA scaffolds were put in a 24-well plate and sterilized using UV light in a tissue culture hood. ES cells were seeded on the PLGA scaffolds and grown for desired days. NIH 3T3 cells were seeded on the PLGA scaffolds and grown for desired days. The PLGA scaffolds containing the cells were stacked layer-by-layer and placed in a sterile container with the culture medium, and a contact pressure of 55 kPa (8 psi) was applied. The whole system was placed in the pressure chamber for CO₂ fusion. At 37° C., an ISCO 500C high-pressure syringe pump was used to deliver and control the CO₂ pressure in the chamber. The CO₂ pressure was increased to 0.69 MPa (100 psi) at an approximate rate of 0.14 MPa (20 psi) per min, and maintained at 0.69 MPA for 10 min, followed by a slow-pressure release at 0.02 MPa (3 psi) per min. After CO₂ fusion, the assembled cell-scaffold constructs were cultured to form complex architecture.

The complicated tissue can be divided into several functional elements and each element can be developed by seeding certain types of cells on scaffolds and differentiating them to a desired degree. The method provides a feasible process to assemble these functional elements into a tissue complex without cell damage. The growth factors to stimulate tissue morphogenesis can be incorporated into the scaffolds and released to the cells tissue-like morphogenesis without loss of bioactivity. The cell types in the scaffold can be pre-determined and pre-developed, and the arrangement of cell-scaffolds can be controlled during assembly.

### EXAMPLE 9

Polymer Fusing in the Presence of Viable Cells and Biomolecules

The PLGA scaffolds containing cells and/or biomolecules were stacked layer-by-layer and placed in a sterile container with the culture medium, and a contact pressure of 55 kPa (8 psi) was applied. The whole system was placed in the pressure chamber for CO₂ fusion. At 37° C., an ISCO 500C high-pressure syringe pump was used to deliver and control the CO₂ pressure in the chamber. The CO₂ pressure was increased to 0.69 MPa (100 psi) at an approximate rate of 0.14 MPa (20 psi) per min, and maintained at 0.69 MPa for 10 min, followed by a slow-pressure release at 0.02 MPa (3 psi) per min. After CO₂ fusion, the assembled cell-scaffold constructs were cultured to form complex architecture.

The method provides a platform to assemble biomedical devices loaded with cells and biomolecules, including, but not limited to, CD ELISA, biochips, and biosensors.

### EXAMPLE 10

Methods of Incorporating Bioactive Biomolecules in Micro-/Nano-Scale Devices

Biomolecules in the form of a powder or solution were contacted with polymer micro/nanostructures, and exposed to CO₂ at a biologically permissive temperature (e.g., 37° C.) and certain pressure (depending on the polymer) for a period of time. Suitable temperatures, pressures, and times can be determined by routine experimentation. Biomolecules diffused into the polymer substrate, and the depth can be controlled by temperature, CO₂ pressure, and exposure time. After the CO₂ was released, the biomolecules were entrapped in the substrate and can be released in a controlled manner in the final applications.

The incorporation efficiency of DNA or protein can be higher than 90%. The bioactivity of DNA and protein was higher than 90%.

### EXAMPLE 11

Assembly in vitro Differentiated Stem Cells on Polymer Scaffolds to Form 3-D Constructs

Stem cells would be first differentiated to desired cell cell types individually, and then formed into functional tissue. For example, stem cells on polymer scaffolds would be induced to endothelial cells and smooth muscle cells. The differentiated endothelial cell-scaffold, smooth muscle cell-scaffold, and fibroblast-scaffold would be stacked layer-by-layer and assembled to form 3-D blood vessel-like constructs.

ES or hMSC would be seeded on the PLGA or PCL scaffolds. They would be grown in differentiation medium for 20 days to form desired multi-type tissue-like structure, respectively. These scaffolds containing cells would be stacked layer-by-layer and placed in a sterile container with the culture medium, and an appropriate contact pressure would be applied (e.g., about 55 kPa (8 psi)). The whole system would be placed in the pressure chamber for CO₂ fusion. At about 37° C., an ISCO 500C high-pressure syringe pump would be used to deliver and control the CO₂ pressure in the chamber. The CO₂ pressure would be increased to an appropriate level (e.g., about 0.69 MPa (100 psi)) at an appropriate rate (e.g., approximately 0.14 MPa (20 psi) per min), and maintained at an appropriate level (e.g., about 0.69 MPa) for an appropriate length of time (e.g., about 10 min), followed by a slow-pressure release (e.g., about 0.02 MPa (3 psi) per min). After CO₂ fusion, the assembled cell-scaffold constructs would be cultured to form a 3-D construct.

Desired multi-type morphology can be formed and assembled into a large amount of cell-scaffold simultaneously.

### EXAMPLE 12

Methods of Fabricating Hierarchical Tissue with Precise Cell Organization

Microstructures would be patterned and fabricated into PLGA films by using photolithography and micro-embossing (Yong Yang et al., Biomaterials, 2004, which is incorporated herein by reference). Cells would be seeded on the well-defined PLGA scaffolds. They would be grown to form desired tissue-like structure, respectively. These scaffolds containing cells would be stacked layer-by-layer and placed in a sterile container with the culture medium, and an appropriate contact pressure would be applied (e.g.;, about 55 kPa (8 psi)). The whole system would be placed in the pressure chamber for CO₂ fusion. At 37° C., an ISCO 500C high-pressure syringe pump would be used to deliver and control the CO₂ pressure in the chamber. The CO₂ pressure would be increased to an appropriate level, (e.g., about 0.69 MPa (100 psi)) at an appropriate rate (e.g., approximately 0.14 MPa (20 psi) per min), and maintained at the appropriate level (e.g., about 0.69 MPa) for an appropriate length of time, (e.g., about 10 min), followed by a slow-pressure release (e.g., about 0.02 MPa (3 psi) per min). After CO₂ fusion, the assembled cell-scaffold constructs would be cultured to form 3D construct.

Well defined microarchitectures and controlled 3-D configurations of multiple cell types would be made.

The cells would be seeded onto a large amount of cell scaffolds directly and simply by gravity. The cells in the scaffolds would be controlled by well defined structure and grow or differentiate to a desired morphology. The multiple cell-scaffolds would form well-defined 3-D structures simultaneously by CO₂ assisted assembly without loss of cell viability and function.

### EXAMPLE 13

Low Temperature and Low Pressure Fusion of Cell/Scaffold Constructs in a Cell Culture Medium.

The scaffolds containing cells were stacked layer-by-layer and placed in a sterile container with the culture medium, and a contact pressure of 55 kPa (8 psi) was applied. The whole system was placed in the pressure chamber for CO₂ fusion. At 37° C., an ISCO 500C high-pressure syringe pump was used to deliver and control the CO₂ pressure in the chamber. The CO₂ pressure was increased to 0.69 MPa (100 psi) at an approximate rate of 0.14 MPa (20 psi) per min, and maintained at 0.69 MPa for 10 min, followed by a slow-pressure release at 0.02 MPa (3 psi) per min. After CO₂ fusion, the assembled cell-scaffold constructs were returned to incubator.

The process is biologically benign and biologically permissive. It can keep the cell viability and bioactivity of proteins and DNAs higher than 90%

### EXAMPLE 14

Biologically Permissive Assembly of Polymers Containing 8 Layer Cells, 2 Proteins, and/or 2 DNAs.

By increasing CO₂ pressure, the processing/assembly temperature was decreased to a biologically permissive level. The CO₂ pressure used has little effect on the viability/activity of the cells, proteins, and DNAs studied. By following the procedure described in Example 13, these biomolecules/cells were integrated into the assembly process.

Cells, proteins, and DNAs, or combinations thereof could be integrated into pre-designed micro/nanoscale polymeric devices. The cells and biologically active molecules maintain their bioactivity and the original micro/nanoscale features are also maintained.

### EXAMPLE 15

Incorporation of Proteins and DNA into Packaging or Assembly of Micro/Nano Devices.

Proteins and DNAs were pre-loaded into the microstructure or nanostructure following the same procedure of assembling cells/scaffold constructs as described in Example 13.

The process is biologically benign and biologically permissive and will maintain the bioactivity of the proteins and DNAs. The bioactivity of the proteins and DNAs was maintained higher than 90%.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method of making a three dimensional tissue scaffold comprising:
providing at least two scaffold layers;
seeding the scaffold layers with cells, biologically active molecules, or combinations thereof;
stacking the at least two scaffold layers; and
bonding the at least two scaffold layers using a gas in the absence of a solvent or an adhesive into the three dimensional scaffold.

2. The method of claim 1 wherein the cells, biologically active molecules, or combinations thereof are selected from cells, proteins, DNA, or combinations thereof.

3. The method of claim 1 wherein the gas is selected from carbon dioxide, methane, ethane, water, nitrogen, oxygen, chlorofluorocarbon, or combinations thereof.

4. The method of claim 1 wherein bonding is performed at a gas pressure less than about 10 MPa.

5. The method of claim 1 wherein the bonding is performed at a contact pressure less than about 1 MPa.

6. The method of claim 1 wherein the bonding is performed in less than about 4 hrs.

7. The method of claim 1 wherein the bonding is performed at a temperature less than about 70° C.

8. The method of claim 1 wherein at least one cell scaffold layer includes nanosize or micronsize features, and wherein the nanosize or micronsize features are retained after bonding.

9. A three dimensional scaffold comprising:
a plurality of scaffold layers seeded with cells, biologically active molecules, or combinations thereof, the plurality of scaffold layers stacked one on top of another and bonded together in the absence of a solvent or an adhesive.

10. The three dimensional scaffold of claim 9 wherein the cells, biologically active molecules, or combinations thereof are selected from cells, proteins, DNA, or combinations thereof.

11. The three dimensional scaffold of claim 9 wherein at least one scaffold layer includes nanosize or micronsize features, and wherein the nanosize or micronsize features are retained after bonding.

12. A method for incorporating cells, biologically active molecules, or combinations thereof in a scaffold layer comprising:
providing a polymeric scaffold layer;
contacting the scaffold layer with cells, biologically active molecules, or combinations thereof; and
exposing the scaffold layer and the cells, biologically active molecules, or combinations thereof to a gas at a biologically permissive temperature, and at a biologically permissive pressure for a sufficient time so that the biologically active molecule diffuses in the scaffold layer.

13. The method of claim 12 wherein the cells, biologically active molecules, or combinations thereof are in the form of a powder or a solution.

14. The method of claim 12 wherein the cells, biologically active molecules, or combinations thereof are selected from cells, proteins, DNA, or combinations thereof.

15. A scaffold layer incorporating cells, biologically active molecules, or combinations thereof comprising:
a polymeric scaffold layer having cells, biologically active molecules, or combinations thereof diffused into the polymeric scaffold layer.

16. The scaffold layer of claim 15 wherein the cells, biologically active molecules, or combinations thereof are selected from cells, proteins, DNA, or combinations thereof.

17. A method of making synthetic tissue comprising:
seeding at least two scaffold layers with cells, biologically active molecules, or combinations thereof;
stacking the at least two scaffold layers;
bonding the at least two scaffold layers using a gas in the absence of a solvent or an adhesive into a three dimensional scaffold construct; and
culturing the three dimensional scaffold construct to form the synthetic tissue.

18. The method of claim 17 wherein the cells, biologically active molecules, or combinations thereof are selected from cells, proteins, DNA, or combinations thereof.

19. Synthetic tissue comprising:
a three dimensional scaffold construct comprising a plurality of scaffold layers seeded with cells, biologically active molecules, or combinations thereof, the plurality of scaffold layers stacked one on top of another and bonded together in the absence of a solvent or an adhesive, the three dimensional construct cultured to form the synthetic tissue.

20. The method of claim 19 wherein the cells, biologically active molecules, or combinations thereof are selected from cells, proteins, DNA, or combinations thereof.

21. A method of constructing tissue with complex architecture comprising:
seeding at least two scaffold layers with cells, biologically active molecules, or combinations thereof;
stacking the at least two scaffold layers;
bonding the at least two scaffold layers using a gas in the absence of a solvent or an adhesive into a three dimensional scaffold construct; and
culturing the three dimensional scaffold construct to form the complex architecture.

22. The method of claim 21 wherein the cells, biologically active molecules, or combinations thereof are selected from cells, proteins, DNA, or combinations thereof.

23. Synthetic tissue having a complex architecture comprising:
a three dimensional scaffold construct comprising a plurality of scaffold layers seeded with cells, biologically active molecules, or combinations thereof, the plurality of scaffold layers stacked one on top of another and bonded together in the absence of a solvent or an adhesive, the three dimensional construct cultured to form the complex architecture.

24. The method of claim 23 wherein the cells, biologically active molecules, or combinations thereof are selected from cells, proteins, DNA, or combinations thereof.
